(19) United States
(12) Patent Application Publication
(10) Pub. No.: US 2022/0226233 A1
(43) Pub. Date: Jul. 21, 2022

(54) Dental Composition Comprising Potassium Nitrate and Propolis

(71) Applicant: SERVIDENCE INC., Saint-Hyacinthe (CA)

(72) Inventor: Jacques Veronneau, Saint-Hyacinthe (CA)

(21) Appl. No.: 17/648,612

(22) Filed: Jan. 21, 2022

Related U.S. Application Data
(60) Provisional application No. 63/139,988, filed on Jan. 21, 2021.

(51) Int. Cl. A61K 8/98; A61Q 11/00; A61K 8/19

(52) U.S. Cl. CPC A61K 8/988; A61K 8/19; A61Q 11/00

(57) ABSTRACT

The present invention provides a dental formulation for the prevention and treatment of dental caries and dental hypersensitivity. The dental formulation is a toothpaste formulation comprising 10% potassium nitrate and 0.5% of propolis; the propolis providing occlusion of tubules and the potassium nitrate providing depolarization of nerve membranes.

### CROSS-REFERENCE TO RELATED APPLICATIONS

The present patent application claims the benefits of priority of U.S. Patent Application No. 63/139,988, entitled “Dental composition comprising potassium nitrate and propolis”, and filed at the United States Patent and Trademark Office on Jan. 21, 2021, the content of which is incorporated herein by reference.

### FIELD OF THE INVENTION

The present invention generally relates to dental compositions for the treatment and prevention of dental hypersensitivity.

### BACKGROUND OF THE INVENTION

Dentin hypersensitivity (DH) has been defined as a “short, sharp pain arising from exposed dentin in response to stimuli typically thermal, evaporative, tactile, osmotic or chemical and which cannot be ascribed to any other form of dental defect or pathology”. DH is one of the most commonly occurring clinical dental conditions at the international level. This problem is prevalent especially in adults who have lost some of the normal protective enamel sheathing on tooth surfaces because of erosion, abrasion, caries, chipping of the enamel, or recession of the gingiva that has exposed tooth dentin or cementum. Brännstrom and Astroem described the mechanism for hypersensitivity based on the “hydrodynamic theory. Hence, the treatment should be aimed at regaining the original state of the dentin, either by occluding the tubules or by gaining control over the neural elements in the pulp tissue that would weaken the stimulatory effects. Available products against DH are generally configured to have one of two specific modes of administration: they're either applied professionally or at-home. These modalities may bring the challenge of partial or complete tubule obliteration, of alteration of sensory activity within the pulp or of both.

Treatment of DH is still a real challenge since most of the dentists are not convinced of the clinical efficacy of the available treatments. Several desensitized products are not scientifically evaluated and related marketing with commercials claims may even be false. There is thus a need to develop other new treatments or products to relieve DH symptoms. Taking this in consideration, there is an increasing interest for natural products to resolve health problem. More specifically, there is an increased need for an evidence based and natural at-home solution to address patients unsatisfied in regard of DH effect by using actual commercial offer.

To reduce DH symptoms, consumers may find fluoride containing toothpaste. Strontium, an original component of Glaxo Smith Kline® product named Sensodyne® is very popular and can use 5% potassium nitrate in various products of the brand. Sensitive Pro Relief® from Colgate Palmolive® uses some arginine-based in its formulation. Arm and Hammer®, with its main brand name Pro-Sensitive®, use 5% of potassium nitrate and some fluoride. Accordingly, most the previous desensitizing agents were mixed together in some clinical studies to assess the potential synergy of the modes of action. After almost 100 years of research, the clinical management of DH continues to be largely empirical in nature and very challenging to the clinicians. In fact, although this condition is painful and frequent for patients, the main problem lies in the fact that DH management and therapeutic products are not always effective. Results of Canadian consensual document on DH established that the majority of dental respondents reported that they lacked confidence in managing their patients' pain. It seems that dentists are not satisfied with the more common treatment methods against DH. Unfortunately, several reports on DH treatments have appeared unserious and associated more with the sharing of testimonies. Similarly, the assumptions of some desensitizing toothpastes on the prevention of caries seem to be unfounded. In describing the problem related to the treatment of DH, it was highlighted in particular the current scientific empty vacuum of numbers of desensitizing products with regard to the commercial claims included in their marketing.

So there continues to be a need to develop new treatments or products to allow better relief of HD symptoms.

Propolis is not listed as a formal agent in most DH treatment reviews. Also, the previous clinical studies suggesting and validating propolis as DH desensitized agent, proposed mainly a professional administration and gave almost no mention of the adequate concentration to use. By the time of preparation, in 1974, Hodosh did not randomize his tests with alternatively potassium nitrate concentrations of 1%, 2%, 5%, 10%, and 15%.

### SUMMARY OF THE INVENTION

The aforesaid and other objectives of the present invention are realized by generally providing a dental formulation, such as a toothpaste, for the prevention and treatment of dental caries and dentinal hypersensitivity, the formulation comprising propolis as a first active agent and potassium nitrate as a second active agent. The propolis proportion may be from about 0.25 wt. % to about 2.00 wt. %. In a further aspect, the propolis proportion is about 0.50 wt. %. The formulation may further comprise at least a carrying agent, a moisturizing agent, a thickening agent, an abrasive agent, a flavor agent, a surfactant agent and a foaming agent.

In another aspect of the invention, the formulation comprises water, barbadensis leaf juice, propolis, hydroxyethyl cellulose, magnesium aluminum silicate, calcium carbonate, lemon flavor, sodium bicarbonate and sodium lauryl sulfoacetate.

In yet another aspect of the invention, the potassium nitrate proportion is from about 5 wt. % to about 10 wt. %. The potassium nitrate proportion is about 5 wt. % in another embodiment wherein it is about 10 wt. % in another embodiment.

In another aspect of the invention, the dental formulation for the prevention and treatment of dental caries and dentinal hypersensitivity comprises the following raw materials:

about 34.07 wt. % of water;

about 31 wt. % of sorbitol;

about 18 wt. % of hydrated silica;

about 10 wt. % of potassium nitrate;

about 1.75 wt. % of glycerin;

about 1 wt. % of PVP;

about 1 wt. % of CI 77891;

about 0.855 wt. % of sodium lauroyl sarcosinate;

about 0.8 wt. % of cellulose gum;

about 0.5 wt. % of aroma;

about 0.5 wt. % of propolis;

about 0.3 wt. % of xanthan gum;

about 0.2 wt. % of sodium saccharin; and

about 0.02174 wt. % of limonene.

The features of the present invention which are believed to be novel are set forth with particularity in the appended claims.

### DETAILED DESCRIPTION OF THE PREFERRED EMBODIMENT

A novel dental formulation comprising potassium nitrate and propolis will be described hereinafter. Although the invention is described in terms of specific illustrative embodiment(s), it is to be understood that the embodiment(s) described herein are by way of example only and that the scope of the invention is not intended to be limited thereby.

As used herein % or wt. % means % by weight as compared to the total weight percent of the phase or composition that is being discussed unless otherwise indicated.

By “about”, it is meant that the value of weight % can vary within a certain range depending on the margin of error of the method or device used to evaluate such weight %. A margin of error of 10% is generally accepted.

First, the usefulness of 10% of potassium in toothpaste formulations as a neural desensitization against DH was tested. The goal being to explore modernly the testing of a 10% toothpaste formulation as desensitizing agent and estimate the effect magnitude. The test was designed to observe the effect with cross-over treatments. Twenty patients with DH symptoms were voluntary recruited from two Quebec dental clinics. The criteria for entering the test was a formal dissatisfaction about the perceived effect against their sensitivity (hot/cold food and drink, air, tooth brushing, and sweet and sour food) with their current commercial toothpaste. To be accepted, participants had to provide a score of 7 or less on 10 in which 0 indicated no effects and 10 indicated ideal effects from their current commercial toothpaste against DH. The majority of the patients reported a prior use of Sensodyne® from Glaxo Smith Kline® and a few of them reported a use of Sensitive Pro Relief® from Colgate Palmolive®. The two above-mentioned toothpastes are formulated with 5% of potassium nitrate. All recruited patients reported suffering of DH for an average of 11 years. Their dissatisfaction score prior to the test had an average score of perceived effect of 5.6 on 10. This score expresses a residual lack of ideal effect of about 4.4 out of 10. Participants were then asked to brush twice a day with a formulation comprising 10% of potassium nitrate. Results showed that a positive effect was perceived after 4.1 days on average. From the total of 20 participants, the perceived effectiveness was 7.3 out of 10. Increasing the concentration from 5% to 10% thus increased the effectiveness by 17% compared the previously used commercial toothpastes. All patients further confirmed an interest in switching to the toothpaste formulation comprising 10% potassium nitrate in the future.

Next, the minimal propolis concentration to have a clinical desensitized effect using an at-home toothpaste vehicle was tested. It is known that pain is a subjective experience and depends on various factors, such as the psychological profile of the individual, his/her previous pain experiences and levels of anxiety. Other aspects that can make the interpretation of alleviating effects of desensitizing agents difficult are the Hawthorne effect. To prevent those potential bias, it was decided to adopt a small randomized control trial as a pilot test. A total of seven different propolis concentrations were tested, though only the results for the first three concentrations are relevant. Ten patients were then recruited that used a commercial toothpaste (mostly Sensodyne®) against DH. The patients were asked to stop the use of the commercial toothpaste until their symptoms were back. All patients were blinded to the exact concentration of propolis and randomly allocated a formulation comprising said concentration of propolis. The first five patients were exposed to three propolis concentrations: 0.25%, 0.50% and 0.75%. Each patient was exposed to all three concentrations. They were asked to qualify, out of 10, the effect of the propolis exposure compared to their actual commercial toothpaste against DH. The score of 10 meant no pain and 0 the highest level of pain. The new toothpaste formulations were tested for three weeks. The concentrations of 0.25% and 0.75% had no reported effect. Formulations with propolis at 0.50% had a significant effect (between 7 and 8 out of 10) and was judged with more impact by patients compared to the commercial toothpastes. Surprisingly, no dose dependent response was observed.

Given the outcome of two studies related to potassium nitrate at 10% and to propolis at 0.5% both against DH symptoms, a test for the potential synergy and complementarity of both ingredients together was to be undertaken. Testing both treatment approaches is meant to reduce pain by targeting the pulpal nerves directly, typically with potassium nitrate, and at the same time by occluding the dentin tubules with propolis. Again, an observational test with cross-over treatments was used. Ten patients with severe DH symptoms were voluntary recruited from two Quebec dental clinics. Recruiting criteria for the test was a formal dissatisfaction about the perceived of the effect of the commercially used toothpaste of the patients against their sensitivity (hot/cold food and drink, air, tooth brushing, and sweet and sour food). To be accepted, participants had to provide a score of 7 or less out of 10, in which 0 indicated no effect and 10 indicated ideal effects against DH. Furthermore, patients with severe abrasion condition, with pain during orthodontic adjustments and patients under whitening treatment leading to pain were recruited. The majority of them reported a prior use of Sensodyne® from Glaxo Smith Kline® and a few of them, using Sensitive Pro Relief® from Colgate Palmolive®. Their dissatisfaction had an average score of perceived effect of 4.6 out of 10. This score express a residual lack of ideal effect of about 5.4 out of 10. Participants were then asked to brush twice a day with a formulation comprising 10% of potassium nitrate and 0.5% of propolis. Results showed that an effect was perceived after 2.1 days on average. The total of 10 participants gave an average score of 8.2 out of 10, which is an improvement of 36% compared the previous commercial toothpastes effect perception. This increasing of the concentration reduced the ultimate effect goal of 10, by 3.6 units. All patients confirmed their interested to adopt this toothpaste in the future.

Example 1 provides a representative toothpaste formulation of the invention. The formulation comprises 0.50% of propolis and 10% of potassium nitrate.

| INGREDIENTS | % wt |
|---|---|
| Water | 34.07 |
| Sorbitol | 31 |
| Hydrated silica | 18 |
| Potassium nitrate | 10 |
| Glycerin | 1.75 |
| PVP | 1 |
| Cl 77891 | 1 |
| Sodium lauroyl sarcosinate | 0.855 |
| Cellulose gum | 0.8 |
| Aroma | 0.5 |
| Propolis | 0.5 |
| Xanthan gum | 0.3 |
| Sodium saccharin | 0.2 |
| Limonene | 0.02174 |

This combination of propolis and potassium nitrate in such concentrations in toothpastes may provide an alternative to patients suffering from severe DH.

Furthermore, the tested toothpaste formulations had the following characteristics. Tested with a PHmetry test diluted at 25%, the pH value of each formulation varied between 6 to 8. A densimeter test revealed a density of about 1.35 g/mL for each formulation. Both tests of aerobic mesophilic flora and moulds and yeasts resulted in under 100 Ufc/g as per rapid microbiological method and/or UNE/EN/ISO 21149:2017 and UNE/EN/ISO 16212:2017. More so, there was an absence of pathogenic microorganisms in the formulations from the tests of absence/presence, rapid microbiological method and/or UNE/EN/ISO 21150:2016, UNE/EN/ISO 22717:2016, UNE/EN/ISO 22718:2016 and UNE/EN/ISO 18416:2016. Additionally, each toothpaste formulation had the following characteristics based on sensorial testing methods: they had a pasty texture, their color was a slightly yellowish white and they tasted like sweet mint. These characteristics are further proof that the different formulations, representing different aspects of the invention, are well suited for at-home usage.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) A dental formulation for the prevention and treatment of dental caries and dentinal hypersensitivity, the formulation comprising propolis as a first active agent and potassium nitrate as a second active agent.

2) The dental formulation of claim 1, wherein the propolis proportion is from about 0.25 wt. % to about 2.00 wt. %.

3) The dental formulation of claim 2, wherein the propolis proportion is about 0.50 wt. %.

4) The dental formulation of claim 1, the formulation further comprising at least a carrying agent, a moisturizing agent, a thickening agent, an abrasive agent, a flavor agent, a surfactant agent and a foaming agent.

5) The dental formulation of claim 4, the formulation comprising water, barbadensis leaf juice, propolis, hydroxyethyl cellulose, magnesium aluminum silicate, calcium carbonate, lemon flavor, sodium bicarbonate and sodium lauryl sulfoacetate.

6) The dental formulation of claim 1, wherein the potassium nitrate proportion is from about 5 wt. % to about 10 wt. %.

7) The dental formulation of claim 6, wherein the potassium nitrate proportion is about 5 wt. %.

8) The dental formulation of claim 6, wherein the potassium nitrate proportion is about 10 wt. %.

9) A dental formulation for the prevention and treatment of dental caries and dentinal hypersensitivity, comprising:
about 34.07 wt. % of water;
about 31 wt. % of sorbitol;
about 18 wt. % of hydrated silica;
about 10 wt. % of potassium nitrate;
about 1.75 wt. % of glycerin;
about 1 wt. % of PVP;
about 1 wt. % of CI 77891;
about 0.855 wt. % of sodium lauroyl sarcosinate;
about 0.8 wt. % of cellulose gum;
about 0.5 wt. % of aroma;
about 0.5 wt. % of propolis;
about 0.3 wt. % of xanthan gum;
about 0.2 wt. % of sodium saccharin; and
about 0.02174 wt. % of limonene.
